(19) United States
(12) Patent Application Publication
(10) Pub. No.: US 2018/0325960 A1
(43) Pub. Date: Nov. 15, 2018

(54) ENHANCED POSTNATAL ADHERENT CELL, AND USE FOR SAME

(71) Applicant: CHA BIOTECH CO., LTD., Gangnam-gu (KR)

(72) Inventor: Hye Sun KIM, Seongnam-si (KR)

(73) Assignee: CHA BIOTECH CO., LTD., Gangnam-gu (KR)

(21) Appl. No.: 15/755,848

(22) PCT Filed: Aug. 26, 2016
(86) PCT No.: PCT/KR2016/009552

§ 371 Date: Feb. 27, 2018

(30) Foreign Application Priority Data
Aug. 28, 2015 (KR) 10-2015-0121747
Aug. 26, 2016 (KR) 10-2016-0109504

(51) Int. Cl. A61K 35/50; C12N 5/0775; C12N 5/077; C12N 5/078; A61P 25/28

(52) U.S. Cl. CPC A61K 35/50; C12N 5/0667; A61P 25/28; C12N 5/0643; C12N 5/0655; C12N 5/0656

(57) ABSTRACT

The present invention relates to an enhanced postnatal adherent cell (ePAC), a method for producing same, and a composition and a cell therapeutic agent having same as an active ingredient.

### TECHNICAL FIELD

The present disclosure relates to enhanced postnatal adherent cells (ePACs), a preparation method thereof, and a cell therapeutic agent including the same as an active ingredient.

### BACKGROUND ART

A cell therapeutic agent is a drug used for the purpose of treatment, diagnosis, or prevention through an action of proliferating or selecting living autologous, allogenic, or xenogenic cells ex vivo or changing biological characteristics of cells in order to restore functions of cells and tissues. The cell therapeutic agents may be classified into somatic cell therapeutic agents and stem cell therapeutic agents according to the kind and the degree of differentiation of cells to be used, and the stem cell therapeutic agents may be classified into embryonic stem cell therapeutic agents and adult stem cell therapeutic agents. The stem cell therapeutic agents have received attention for an ideal reason to treat a patient's disease and to improve symptoms by helping regeneration of damaged cells using stem cells having self-renewal capacity and multipotency. However, studies on stem cells have difficulties in terms of ethical issues and acquisition of the desired number of cells. Although embryonic stem cells have superior performance as compared with adult stem cells, there is a negative aspect in terms of bioethics. Adult stem cells have disadvantages that donors suffer from pains during the extraction and isolation of stem cells and a separation efficiency of stem cells is low.

Meanwhile, the placenta is a tissue which is rich and discarded as medical waste after birth. Placenta-derived cells are characterized in that since they are separated and extracted from the removed placenta, ethical issues may be avoided and a large amount of desired cells may be easily obtained. Therefore, the placenta provides an ethically non-controversial and easily accessible source of cell therapeutic agents for experimental and clinical applications. Accordingly, if the placenta-derived cells, which are free of ethical problems and are produced in a large amount, may perform the functions of existing stem cells, they may be used as excellent cell therapeutic agents.

Under this background, the present inventors isolated novel multifunctional cells from the placenta, the cells having marker characteristics different from those of known placenta-derived stem cells and excellent proliferation ability and differentiation ability, thereby completing the present invention.

### DETAILED DESCRIPTION OF THE INVENTION

### Technical Problem

An aspect provides placenta-derived stem cells having novel characteristics.

Another aspect provides cell populations including the placenta-derived stem cells having novel characteristics.

Still another aspect provides a method of preparing the placenta-derived stem cells having novel characteristics.

Still another aspect provides a composition and a cell therapeutic agent including the placenta-derived stem cells having novel characteristics.

### Technical Solution

An aspect provides enhanced postnatal adherent cells. The enhanced postnatal adherent cells may have one or more characteristics selected from the group consisting of:

(a) maintaining a morphology of adherent fibroblasts during subculture;

(b) having ability to differentiate into adipocytes, osteocytes, or chondrocytes;

(c) secreting one or more proteins selected from the group consisting of VEGF, TGF-β1, IL-6, progranulin, follistatin, angiostatin, MMP2, MMP10, TRAIL R2, and MMP3,

(d) having surface antigen characteristics of HLA-G−, CD34−, MIC A/B+, CD200+, CD61+, CD130+, CD321+, SSEA4+, and CD338+;

(e) having a high expression level of one or more genes selected from the group consisting of COL3A1, IGFBP5, PRNP, and MT1A, as compared with bone marrow-derived stem cells;

(f) having a low expression level of one or more genes selected from the group consisting of COL1A1, COL1A2, TPM2, TAGLN, CYP1B1, CXCL12, and PENK, as compared with bone marrow-derived stem cells;

(g) having a high expression level of one or more proteins selected from the group consisting of LIN28B, FERMT3, RAB27B, and PEG10, as compared with bone marrow-derived stem cells; and

(h) having a low expression level of HYOU1 or GLIPR1 protein, as compared with bone marrow-derived stem cells.

The term “enhanced postnatal adherent cells (ePACs)”, as used herein, refer to cells that are separated from the placenta, and have a property of adhering to and growing on the surface of a culture plate, and have excellent proliferation ability and ability to differentiate into cells of various tissues. The ePACs of the present disclosure may be also called ‘postnatal adherent cells’.

The term “placenta”, as used herein, refers to an organ that develops for a fetus during pregnancy of a mammal, and includes amnion, chorion, or decidua. Amnion is a clear membrane surrounding a fetus and containing amniotic fluid. In the amnion, stem cells of the fetus may exist. Deciduas is a membrane formed as a result of a process in which the epithelial cells of the uterus are modified so that a fertilized egg becomes implanted in the uterine wall. In the deciduas, stem cells of the mother may exist. Chorion is a membrane between amnion surrounding the fetus or amniotic fluid and deciduas, and develops from a fertilized egg to constitute a part of egg membrane.

The placenta may be specifically a human placenta, and specifically, a placenta separated from the human body.

In the present disclosure, the placenta may be specifically amnion, chorion, or trophoblast. That is, the ePACs of the present disclosure may be specifically adherent cells that are isolated from the amnion, chorion, or trophoblast. More specifically, the cells may be cells that are isolated from the amnion. Most specifically, the cells may be cells that are isolated from the amnion of the human placenta separated from the human body.

The ePACs may be derived from a fetus and/or a mother (i.e., the ePACs may have the genotype of the fetus or mother), and specifically, derived from the fetus.

The ePACs of the present disclosure may have an adherent fibroblast morphology during subculture. Specifically, the cells of the present disclosure may have the property of cells requiring adhesion to the surface to grow in vitro. In a specific embodiment, the cells of the present disclosure may exhibit a specific morphology of spindle-shaped fibroblasts. Further, the cells of the present disclosure may have an average diameter of 20 μm or less until passage 18, and an average diameter of 13 μm to 14 μm at early passages, i.e., passage 1 to passage 6.

The ePACs may differentiate into adipocytes, osteocytes, chondrocytes, etc. For example, the cells may be induced to differentiate into particular cell lineages including adipocytes, chondrocytes, osteoblasts, hematopoietic cells, myocytes, vascular cells, neuronal cells, or hepatocytes.

The term “differentiation” refers to a process by which cells become more specialized in structure or function during cell growth through division and proliferation, i.e., a process by which cells, tissues, etc. of a living body change in shape or function in order to perform the given task. Determination of differentiation into particular cell lineages may be accomplished by methods well-known in the art, and differentiation into particular cells may be induced through the known methods. Further, the differentiation may be confirmed by measuring changes in cell surface markers (e.g., staining cells with tissue-specific or cell-marker specific antibodies) and morphology using techniques such as flow cytometry or immunocytochemistry, or by examining the morphology of cells using an optical microscope or confocal microscope, or by measuring changes in gene expression using techniques well known in the art, such as PCR and gene-expression profiling.

Further, the ePACs may secrete one or more proteins selected from the group consisting of VEGF, TGF-β1, IL-6, progranulin, follistatin, angiostatin, MMP2, MMP10, TRAIL R2, and MMP3. Specifically, the ePACs may secrete progranulin. More specifically, the ePACs may secrete two or more, three or more selected from the group consisting of VEGF, TGF-β1, IL-6, progranulin, follistatin, angiostatin, MMP2, MMP10, TRAIL R2, and MMP3, or all of them. Most specifically, the ePACs may secrete progranulin, or progranulin and one or more of the other proteins. In a specific embodiment, the cells of the present disclosure may exhibit increased secretion of VEGF and/or IL-6 as the passage increases. Specifically, the ePACs may exhibit increased secretion of VEGF and/or IL-6 as the passage increases until passage 6. Further, the ePACs may exhibit increased secretion of VEGF and/or IL-6 twice or more times from passage 5, as compared with passage 1. In another specific embodiment, the cells of the present disclosure may constantly secrete TGF-β1 and/or progranulin regardless of the passage increase.

Further, the ePACs may further secrete HGF, in addition to VEGF, TGF-β1, IL-6, and/or progranulin. In a specific embodiment, the cells of the present disclosure may constantly secrete HGF regardless of a donor and/or passage.

The ePACs of the present disclosure may exhibit a negative immunological characteristic for HLA-G which is a surface antigen regarding antigen compatibility, and/or a positive immunological characteristic for CD200 which is a surface antigen involved in immune suppression. Further, the ePACs may exhibit positive immunological characteristics for CD200, CD61, CD130, CD321, SSEA4, MIC A/B, and CD338. Further, the ePACs may exhibit a negative immunological characteristic for CD34 which is expressed in hematopoietic stem cells.

In a specific embodiment, the ePACs of the present disclosure may exhibit a high expression level of CD200, as compared with that in other tissues analyzed in the previous studies (umbilical cord blood-derived mesenchymal stem cells do not express CD2000 (PIoSONE, February 2012, vol 7, Issue 2, e31671), and about less than 60% of adherent stromal cells are positive for CD200 as measured by flow cytometry (Korean Patent No. 10-2013-7027706)). Therefore, the ePACs may have excellent immune modulating ability, as compared with other cells. 70% or more of the cells of the present disclosure may maintain the positive immunological characteristic for CD200.

The term “positive”, as used herein, with respect to a cell marker, means that the cell marker exists in a large amount or a high concentration, as compared with that in other cells as a reference. Specifically, any marker is present inside or on the surface of a cell, and therefore, if a cell may be distinguished from one or more other cell types by using the marker, the cell may be positive for the marker. Further, the term “positive” means that cells have signals of higher intensity than a background intensity, for example, cells have the marker in an amount enough to be detectable in a cell-measuring device. For example, cells may be detectably labeled with CD105-specific antibodies, and when signals from these antibodies are detectably stronger than those of a control (e.g., background intensity), the cells are “positive for CD45” or “CD105+”. The term “negative”, as used herein, means that although antibodies specific to a particular cell surface marker are used, the marker cannot be detected, as compared with the background intensity. For example, if a cell cannot be detectably labeled with a CD45-specific antibody, the cell is “negative for CD45” or “CD45-”.

The above immunological characteristics may be determined by common methods known in the art to which the present disclosure pertains. For example, various methods such as flow cytometry, immunohistochemical staining, RT-PCR, etc. may be used. In a specific embodiment, flow cytometry may be performed to confirm that the ePACs of the present disclosure may have negative characteristics for HLA-G and CD34 and positive characteristics for CD200, CD61, CD130, CD321, SSEA4, MIC A/B, and CD338.

Further, the ePACs of the present disclosure may further exhibit negative immunological characteristics for Oct4, Nanog, and/or TRA-1-60 which are markers representing maintenance of progenitor cell properties of embryonic stem cells. In a specific embodiment, flow cytometry may be performed to confirm that the ePACs of the present disclosure may have a negative characteristic for TRA-1-60, and immunocytochemical staining may be performed to confirm that the ePACs of the present disclosure may have a negative characteristic for Oct4 and Nanog.

The ePACs of the present disclosure may further exhibit negative immunological characteristics for CD44, CD73, CD90, and CD105 which are markers having properties of mesenchymal stem cells, and for CD3, CD8a, CD11c, CD19, CD45, and CD56 which are expressed in blood cells. The ePACs of the present disclosure may further exhibit negative immunological characteristics for CD80 and CD86 which are co-stimulators stimulating immune activity, or/and for CD31 which is expressed in vascular endothelial cells.

The cells may further have positive immunological characteristics for HLA-ABC which is a surface antigen regarding antigen compatibility and for CD29 and CD49 which are involved in ability to migrate into damaged tissues. Further, the cells may have a positive immunological characteristic for CD9 which is SSEA4 representing a non-trophoblast. Further, the cells may have positive or partially positive immunological characteristics for CD54, CD106, and CD166 which are surface antigens involved in immune suppression.

The additional immunological characteristics may be specifically determined by flow cytometry.

The ePACs of the present disclosure may have high expression of one or more genes selected from the group consisting of COL3A1, IGFBP5, PRNP, and MT1A, as compared with bone marrow-derived stem cells. Specifically, the ePACs of the present disclosure may have high expression of two or more, or three or more genes selected from the group consisting of COL3A1, IGFBP5, PRNP, and MT1A, and more specifically, all of the genes, as compared with bone marrow-derived stem cells.

Further, the ePACs of the present disclosure may have low expression of one or more genes or proteins selected from the group consisting of COL1A1, COL1A2, TPM2, TAGLN, CYP1B1, CXCL12, PENK, HYOU1, and GLIPR1, as compared with bone marrow-derived stem cells. Specifically, the ePACs of the present disclosure may have low expression of two or more, or three or more genes or proteins selected from the group consisting of COL1A1, COL1A2, TPM2, TAGLN, CYP1B1, CXCL12, PENK, HYOU1, and GLIPR1, and more specifically, all of the genes or proteins, as compared with bone marrow-derived stem cells.

Specifically, a difference in the expression levels of the genes between the cells of the present disclosure and bone marrow-derived stem cells may be twice or higher, and a difference in the expression levels of the proteins therebetween may be 1.5 times or more. The difference in the expression levels may be determined by, for example, comparing the gene and protein expression levels at an mRNA level or at a protein level. The difference in the expression levels may be determined by, for example, microarray or proteomics array.

The ePACs cultured under a hypoxia condition may have increased expression level of one or more genes selected from the group consisting of PGK1, BNIP3, TPI1, ERRFI1, LOC644774, SLC2A3, PLIN2, and TUBB2B, as compared with those cultured under a normoxia condition. Specifically, the ePACs cultured under a hypoxia condition may have increased expression level of two or more, three or more, four or more, five or more, or six or more genes selected from the group consisting of PGK1, BNIP3, TPI1, ERRFI1, LOC644774, SLC2A3, PLIN2, and TUBB2B, or increased expression level of all of the genes, as compared with those cultured under a normoxia condition. A difference in the expression levels may be twice or higher. Specifically, the difference in the expression level of ERRFI1, LOC644774, or SLC2A3 between the cells cultured under the normoxia condition and the cells cultured under the hypoxia condition may be three times or more.

Further, the ePACs cultured under a hypoxia condition may have decreased expression level of one or more genes or proteins selected from the group consisting of SERPINE1, TAGLN, TGM2, IL8, ALDH1A3, and DPP4, as compared with those cultured under a normoxia condition. Specifically, the ePACs cultured under a hypoxia condition may have decreased expression level of two or more, three or more, or four or more genes selected from the group consisting of SERPINE1, TAGLN, TGM2, IL8, and ALDH1A3, or decreased expression level of all of the genes, as compared with those cultured under a normoxia condition. A difference in the expression levels may be twice or higher. Specifically, the difference in the expression level of SERPINE1, TAGLN, IL8, or ALDH1A3 between the cells cultured under the normoxia condition and the cultured under the hypoxia condition may be 2.5 times or more, the difference in the expression level of IL8 may be 4 times or more, and the difference in the expression level of DPP4 protein may be 1.5 times or more.

The difference in the expression levels under the normoxia culture condition and the hypoxia culture condition may be determined by, for example, comparing the gene and protein expression levels at an mRNA level or at a protein level. Further, the difference in the expression levels may be determined by, for example, microarray or proteomics array.

Another aspect provides populations of the ePACs of the present disclosure.

The ePACs included in the cell populations are the same as descried above.

In a specific embodiment, the cell populations may include 50% or more, 60% or more, 70% or more, 80% or more, 90% or more, 95% or more, 98% or more, or 99% or more of the ePACs.

Specifically, the cell populations of the present disclosure may have characteristics of:

(a) maintaining a morphology of adherent fibroblasts during subculture;

(b) having ability to differentiate into adipocytes, osteocytes, or chondrocytes;

(c) secreting one or more proteins selected from the group consisting of VEGF, TGF-β1, IL-6, progranulin, follistatin, angiostatin, MMP2, MMP10, TRAIL R2, and MMP3;

(d) having surface antigen characteristics of HLA-G−, CD34−, MIC A/B+, CD200+, CD61+, CD130+, CD321+, SSEA4+, and CD338+;

(e) having a high expression level of one or more genes selected from the group consisting of COL3A1, IGFBP5, PRNP, and MT1A, as compared with bone marrow-derived stem cells;

(f) having a low expression level of one or more genes selected from the group consisting of COL1A1, COL1A2, TPM2, TAGLN, CYP1B1, CXCL12, and PENK, as compared with bone marrow-derived stem cells;

(g) having a high expression level of one or more proteins selected from the group consisting of LIN28B, FERMT3, RAB27B, and PEG10, as compared with bone marrow-derived stem cells; and

(h) having a low expression level of HYOU1 or GLIPR1 protein, as compared with bone marrow-derived stem cells.

The cells of the cell populations may be specifically derived from amnion. About 50%, about 55%, about 60%, about 65%, about 70%, about 75%, about 80%, about 85%, about 90% or more of the cells of the cell populations of the present disclosure may exhibit a negative immunological characteristic for HLA-G. Further, about 50%, about 55%, about 60%, about 65%, about 70%, about 75%, about 80%, about 85%, about 90% or more of the cells of the cell populations may exhibit a negative immunological characteristic for CD34. For example, 50% or less, 40% or less, 30% or less, 20% or less, 10% or less, 5% or less, or 1% or less of the cells of the cell populations may exhibit a positive immunological characteristic for CD34. Further, about 50%, about 55%, about 60%, about 65%, about 70%, about 75%, about 80%, about 85%, about 90% or more of the cells of the cell populations may exhibit positive immunological characteristics for CD200, CD61, CD130, CD321, SSEA4, and CD338. Specifically, about 70%, about 80%, about 85%, about 90% or more of the cells of the cell populations may exhibit positive immunological characteristics for CD200, CD61, CD130, CD321, SSEA4, and CD338.

Further, about 50%, about 55%, about 60%, about 65%, about 70%, about 75%, about 80%, about 85%, about 90% or more of the cells of the cell populations of the present disclosure may exhibit negative immunological characteristics for Oct4, Nanog, and/or TRA-1-60.

Further, about 20%, about 25%, about 30%, about 35%, about 40%, about 45%, about 50%, about 55%, about 60%, about 65%, about 70%, about 75%, about 80%, about 85%, or about 90% or more of the cells of the cell populations may have high expression of one or more genes or proteins selected from the group consisting of COL3A1, IGFBP5, PRNP, MT1A, LIN28B, FERMT3, RAB27B, and PEG10, as compared with bone marrow-derived stem cells. Further, about 20%, about 25%, about 30%, about 35%, about 40%, about 45%, about 50%, about 55%, about 60%, about 65%, about 70%, about 75%, about 80%, about 85%, or about 90% or more of the cells of the cell populations may have low expression of one or more genes or proteins selected from the group consisting of COL1A1, COL1A2, TPM2, TAGLN, CYP1B1, CXCL12, PENK, HYOU1, and GLIPR1, as compared with bone marrow-derived stem cells.

Further, about 20%, about 25%, about 30%, about 35%, about 40%, about 45%, about 50%, about 55%, about 60%, about 65%, about 70%, about 75%, about 80%, about 85%, or about 90% or more of the cells of the cell populations may express progranulin.

Further, about 50%, about 60%, about 70%, about 80%, about 90%, about 95% or about 98% or more of the cells of the cell populations cultured under a hypoxia condition may have high mRNA or protein expression levels of one or more genes selected from the group consisting of PGK1, BNIP3, TPI1, ERRFI1, LOC644774, SLC2A3, PLIN2, TUBB2B, PODXL, HIST1H1B, and PLEK2, as compared with those cultured under a normoxia condition. Further, about 50%, about 60%, about 70%, about 80%, about 90%, about 95% or about 98% or more of the cells of the cell populations cultured under a hypoxia condition may have low mRNA or protein expression levels of one or more genes selected from the group consisting of PSERPINE1, TAGLN, TGM2, IL8, ALDH1A3, and DPP4, as compared with those cultured under a normoxia condition.

Still another aspect provides a method of preparing ePACs, the method including reacting an amnion tissue separated from a chorionic plate membrane of placenta with an enzyme mixture. The ePACs are the same as described above.

Specifically, the preparation method of the present disclosure may be to prepare the ePACs having the following characteristics of:

(a) maintaining a morphology of adherent fibroblasts during subculture;

(b) having ability to differentiate into adipocytes, osteocytes, or chondrocytes;

(c) secreting one or more proteins selected from the group consisting of VEGF, TGF-β1, IL-6, progranulin, follistatin, angiostatin, MMP2, MMP10, TRAIL R2, and MMP3;

(d) having surface antigen characteristics of HLA-G−, CD34−, MIC A/B+, CD200+, CD61+, CD130+, CD321+, SSEA4+, and CD338+;

(e) having a high expression level of one or more genes selected from the group consisting of COL3A1, IGFBP5, PRNP, and MT1A, as compared with bone marrow-derived stem cells;

(f) having a low expression level of one or more genes selected from the group consisting of COL1A1, COL1A2, TPM2, TAGLN, CYP1B1, CXCL12, and PENK, as compared with bone marrow-derived stem cells;

(g) having a high expression level of one or more proteins selected from the group consisting of LIN28B, FERMT3, RAB27B, and PEG10, as compared with bone marrow-derived stem cells; and

(h) having a low expression level of HYOU1 or GLIPR1 protein, as compared with bone marrow-derived stem cells.

More specifically, the preparation method may be to prepare the ePACs further having a characteristic of secreting progranulin protein. In the preparation method, the cells may further have negative immunological characteristics for Oct4, Nanog, TRA-1-60, and/or CD80.

In a specific embodiment, the amnion tissue may be obtained by separating the chorionic plate membrane from the removed placenta, and then removing the chorion by scraping the separated chorionic plate membrane. The chorionic plate membrane may be separated by, for example, pulling and peeling the chorionic plate membrane from the placenta.

Specifically, the amnion may be obtained by removing blood from the chorionic plate membrane by using Ca/Mg-free DPBS containing gentamicin. In a specific embodiment, the chorionic plate membrane is washed with Ca/Mg-free DPBS containing gentamicin to remove the blood, and then chorion is removed therefrom by scraping, thereby obtaining the amnion.

In the preparation method of the present disclosure, the obtained amnion tissue may be directly reacted with an enzyme mixture, or may be divided into smaller pieces by using sterile scissors, and then reacted with the enzyme mixture. For example, the amnion tissue is divided into smaller pieces (e.g., about 5 mm or less) using sterile scissors, etc., and then the divided cells may be treated with the enzyme mixture.

The enzyme mixture may be an enzyme mixture of various kinds of enzymes or an enzyme reaction solution. The enzyme mixture may dissolve the tissue to separate ePACs from the tissue. The enzyme mixture may specifically include one or more selected from the group consisting of trypsin, dispase, and collagenase. The enzyme mixture may more specifically include all of trypsin, dispase, and collagenase. The enzyme mixture may further include DNase. The enzyme mixture may include water or saline, for example, Hank's balanced salt solution (HBSS) containing collagenase, trypsin, and dispase.

The collagenase may refer to an enzyme that cleaves peptide bonds of collagen, and may include collagenase type I, type II, type III, type IV, or a combination thereof, and specifically collagenase type I. Further, the DNase may be DNA hydrolase (deoxyribonuclease, DNase) I or/and II. A concentration of collagenase in the enzyme mixed solution may be, for example, 0.5 mg/ml to 5 mg/ml, 0.5 mg/ml to 3 mg/ml, 0.8 mg/ml to 2 mg/ml, or 0.8 mg/ml to 1.5 mg/ml, and in a specific embodiment, 1.2 mg/ml. A concentration of trypsin in the enzyme mixed solution may be, for example, 1 mg/ml to 5 mg/ml, 1 mg/ml to 3 mg/ml, 1.5 mg/ml to 2.5 mg/ml, or 1.5 mg/ml to 2 mg/ml, and in a specific embodiment, 1.8 mg/ml. A concentration of dispase in the enzyme mixed solution may be, for example, 0.1 U/ml to 5 U/ml, 0.1 U/ml to 3 U/ml, 0.5 U/ml to 2.5 U/ml, or 0.5 U/ml to 1.5 U/ml, and in a specific embodiment, 1 U/ml. A concentration of DNA hydrolase in the enzyme mixed solution may be, for example, 0.001 mg/ml to 1 mg/ml, 0.001 mg/ml to 0.5 mg/ml, 0.01 mg/ml to 0.25 mg/ml, or 0.01 mg/ml to 0.05 mg/ml, and in a specific embodiment, 25 μg/ml.

In a specific embodiment, reaction of the amnion tissue and the enzyme mixed solution may be allowed under shaking, and the shaking may be performed at about 20° C. to about 40° C., about 30° C. to about 40° C., or about 35° C. to about 40° C., for example, at about 37° C. for about 5 minutes to about 60 minutes or about 10 minutes to about 30 minutes.

Additionally, after reaction of the tissue and the enzyme mixed solution, a process of inactivating the enzyme reaction solution may be further performed, and for example, the enzymatic reaction may be terminated by adding FBS. Further, a method of isolating tissue cells, for example, amniotic cells (i.e., adherent cells) from the enzyme reaction solution may be performed by a common method known in the art. For example, after centrifugation, cells may be isolated by using a cell strainer.

In the method of preparing ePACs according to a specific embodiment of the present disclosure, harvesting of the cell populations may increase yield of ePACs to, for example, 50 times or higher by using the enzyme mixed solution, as compared with yield in a method of not using the enzyme mixed solution.

The method of preparing ePACs of the present disclosure may further include reacting an animal component-free recombinant enzyme with the cells which are recovered after reaction of the amnion tissue and the enzyme mixture.

The isolated cell populations are subjected to adherent culture in a container, and then treated with the animal component-free recombinant enzyme to increase isolation purity of adherent cells. The adherent culture (e.g., P0) of the isolated cell populations in a container (e.g., flask) may include culturing the cell populations in a stem cell culture medium, for example, in a medium to which fibroblast growth factor (FGF-4) and heparin are added. FGF-4 may be added to the medium at a concentration of about 10 ng/ml to about 40 ng/ml, or about 20 ng/ml to about 30 mg/ml, for example, at a concentration of 25 ng/ml. Heparin may be added to the medium at a concentration of about 0.5 μg/ml to about 2 μg/ml, or about 0.5 μg/ml to about 1.5 μg/ml, for example, at a concentration of 1 μg/ml. The medium may further include, for example, fetal bovine serum, and an antibiotic (e.g., penicillin, streptomycin, gentamicin, etc.). In a specific embodiment, a PS-CM medium containing 10% fetal bovine serum, 50 μg/ml of gentamicin, 1 μg/ml of heparin, and 25 ng/ml of FGF-4 may be used. The culture may be performed under a normoxia condition or under a hypoxia condition. The term “normoxia condition” means an oxygen partial pressure of 21%. The term “hypoxia condition” means an oxygen partial pressure lower than a general normoxia condition. The hypoxia condition may be a condition having an oxygen partial pressure of 1% to 15%, 1% to 12%, 1% to 10%, or 1% to 5%, for example, 1%, 2%, 3%, 4%, 5%, 6%, 7%, 8%, or 9%. The culture may be performed, for example, for 2 days to 7 days, or for 3 days to 5 days, and after culture, the animal component-free recombinant enzyme may be treated thereto.

The term “animal component-free enzyme”, as used herein, means that the enzyme is originated from a non-animal, which means that the enzyme is not purified from an animal supply source. The animal component-free enzyme may be originated from recombination, for example, originated from bacteria, yeasts, or plants. The enzyme originated from recombination may mean any enzyme produced by recombinant DNA technology including use of microorganisms, for example, bacteria, viruses, yeasts, plants, etc. The enzyme may be animal component-free recombinant trypsin, for example, recombinant trypsin produced in corn. The animal component-free recombinant trypsin is commercially available, and for example, it may be TrypLE™ Select (GIBCO Invitrogen), TrypLE™ Express (GIBCO Invitrogen), TrypZean™ (Sigma Aldrich), or Recombinant Trypsin Solution™ (Biological Industries). In the method of preparing ePACs of the present disclosure, treatment of the animal component-free recombinant enzyme may not separate dense cell masses and may remain only homogeneous cells after subculture, as compared with non-treatment of the animal component-free recombinant enzyme.

In the method of preparing ePACs according to a specific embodiment of the present disclosure, a passage number of the subculture is not particularly limited, and the passage number may be appropriately selected according to the desired number of proliferating cells. For example, 1 passage to 20 passages, 1 passage to 6 passages, 1 passage, 3 passages, or 6 passages may be performed to obtain the clinically required cumulative number of proliferating cells. Specifically, the passage number may be at least 1 passage to 10 passages.

The method of preparing ePACs according to a specific embodiment of the present invention includes subculturing ePACs obtained as above in a stem cell culture medium, for example, in a medium to which fibroblast growth factor (FGF-4) and heparin are added. The medium to which FGF-4 and heparin are added, and culture conditions are the same as described above. Further, upon subculture, treatment of the animal component-free recombinant enzyme may be also additionally performed as described above. That is, at every stage of subculture before subculture of the cells to the next stage, the cells were treated with the animal component-free recombinant enzyme and harvested to increase purity of the adherent cells. For example, the animal component-free recombinant enzyme may be treated before transferring the cells for P2 at the stage from P1 to P2.

Still another aspect provides ePACs prepared by the above preparation method.

The ePACs are the same as described above.

The ePACs prepared by the method of preparing the ePACs according to a specific embodiment may secrete vascular endothelial growth factor (VEGF), transforming growth factor (TGF)-β1, hepatocyte growth factor (HGF), interleukin-6 (IL-6), progranulin, follistatin, angiostatin, matrix metalloproteinase 2 (MMP2), matrix metalloproteinase 10 (MMP10), TNF-related apoptosis-inducing ligand Receptor2 (TRAIL R2), or matrix metalloproteinase 3 (MMP3) which is specifically effective for neurological diseases, and also have a remarkable ability to migrate to damaged tissues. Therefore, ePACs may be used as a cell therapeutic agent in the treatment of neurological diseases and other diseases on which the above secretory proteins may effectively act.

Still another aspect provides a composition including the ePACs of the present disclosure or the cell populations of the ePACs of the present disclosure.

Still another aspect provides a cell therapeutic agent, or a pharmaceutical composition or agent including the ePACs of the present disclosure, the cell populations thereof, or a culture thereof as an active ingredient.

Still another aspect provides use of the ePACs of the present disclosure, the cell populations thereof, or the culture thereof in the preparation of the cell therapeutic agent, or the pharmaceutical composition or agent.

The ePACs and the cell populations including the ePACs are the same as described above, respectively.

The composition may be a composition for proliferation of the ePACs or differentiation of the ePACs into particular cells.

Further, the ePACs may release proteins that are advantageous for disease treatment and have a remarkable ability to migrate into damaged tissues. Therefore, the composition may be a pharmaceutical composition for the prevention or treatment of various diseases. Specifically, the composition may be a pharmaceutical composition for the prevention or treatment of neurodegenerative diseases. Further, the neurodegenerative diseases may include, for example, one or more selected from the group consisting of Alzheimer's disease, concussion, stroke, Parkinson's disease, Pick's disease, Huntington's disease, progressive supranuclear palsy, multiple sclerosis, amyotrophic lateral sclerosis (ALS), dementia, and traumatic brain injury (TBI).

Therefore, still another aspect provides use of the ePACs, the cell populations thereof, or the culture thereof in the preparation of a drug for treating or preventing a disease, for example, neurodegenerative diseases.

Still another aspect provides a method of treating or preventing a disease, for example, neurodegenerative diseases, the method including administering the ePACs, the cell populations thereof, or the culture thereof as an active ingredient to a subject in need thereof.

A dosage of the pharmaceutical composition according to a specific embodiment may be 1.0×10³ cells/kg (body weight) or subject to 1.0×10¹⁰ cells/kg (body weight) or subject, or 1.0×10⁷ cells/kg (body weight) or subject to 1.0×10⁸ cells/kg (body weight) or subject, based on the adherent cells. However, the dosage may be variously prescribed depending on various factors such as a formulation method, an administration mode, a patient's age, body weight, sex, disease conditions, diet, an administration time, an administration route, an excretion rate, and reaction sensitivity, and those skilled in the art may appropriately adjust the dosage, considering these factors. Administration frequency may be once or twice or more within the clinically allowable range of side effects, and administration may be given to one site or two or more sites. The dosage per kg or per subject for non-human animals may be the same as that for humans, or may be converted from the above-described dosage, for example, based on a volume ratio (e.g., mean value) between organs (heart, etc.) of the human and animal subjects. Animals to be treated according to a specific embodiment may be exemplified by humans and other desired mammals, and specifically, may include humans, monkeys, mice, rats, rabbits, sheep, cows, dogs, horses, pigs, etc.

The pharmaceutical composition according to a specific embodiment may include the adherent cells as an active ingredient and pharmaceutically acceptable carriers and/or additives, and for example, may include sterilized water, physiological saline, a standard buffer (e.g., phosphoric acid, citric acid, or other organic acids), a stabilizer, a salt, an antioxidant (e.g., ascorbic acid, etc.), a surfactant, a suspending agent, an isotonic agent, a preservative, etc. For local administration, the pharmaceutical composition is preferably combined with an organic substance such as a biopolymer, an inorganic substance such as hydroxyapatite, specifically, collagen matrix, a polymer or copolymer of polylactic acid, a polymer or copolymer of polyethylene glycol, and chemical derivatives thereof. When the cell therapeutic agent or the pharmaceutical composition according to a specific embodiment is prepared in an injectable formulation, cell populations may be dissolved in a pharmaceutically acceptable carrier or may be frozen in a solution state in which the cell populations are dissolved.

The pharmaceutical composition according to a specific embodiment may include, if necessary, a suspending agent, a solubilizing aid, a stabilizer, an isotonic agent, a preservative, an adsorption inhibitor, a surfactant, a diluent, an excipient, a pH adjuster, an analgesic agent, a buffer, a reducing agent, an antioxidant, etc., depending upon the administration mode and formulation. In addition to those described above, pharmaceutically acceptable carriers and agents suitable in the present disclosure are described in detail in a literature [Remington's Pharmaceutical Sciences, 19^{th} ed., 1995].

The pharmaceutical composition according to a specific embodiment may be formulated in a unit dosage form or into a multidose container using a pharmaceutically acceptable carrier and/or excipient according to a method that may be easily carried out by those skilled in the art to which the present disclosure pertains. In this regard, the formulation may be in a form of a solution, a suspension, or an emulsion in an oily or aqueous medium, a powder, granules, a tablet, or a capsule.

Still another aspect provides an agent including the ePACs or the cell populations including the same.

In the agent of the present disclosure, the ePACs and the cell populations including the same are the same as described above, respectively.

The agent may be a pharmaceutical agent. The agent may include the ePACs or the cell populations including the same, and may further include adipocytes, osteoblasts, myocytes, neurons, chondrocytes, and myocardiocyte which are differentiated therefrom. The agent may be an agent for oral or parenteral administration. The agent may include a common pharmaceutically acceptable carrier. For example, an injectable formulation may include a preservative, an analgesic agent, a solubilizer, a stabilizer, etc., and a topical formulation may include a base, an excipient, a lubricant, a preservative, etc.

Still another aspect provide a cell therapeutic agent including ePACs or the cell populations including the same as an active ingredient.

In the cell therapeutic agent of the present disclosure, the ePACs and the cell populations including the same are the same as described above, respectively.

The term ‘cell therapeutic agent’, as used herein, refers to cells or tissues that are separated from humans, cultured, and prepared by a specialized method, and is used as a drug for the purpose of treatment, diagnosis, and prevention. Also, the cell therapeutic agent may refer to a drug used for the purpose of treatment, diagnosis, or prevention by a series of actions of proliferating or selecting living autologous, allogenic, or xenogenic cells ex vivo or changing biological characteristics of cells in order to restore functions of cells and tissues.

The ePACs of the present disclosure may be used in various kinds of therapeutic protocols for enhancing, treating, or replacing an organ or a tissue of the body by engraftment, transplantation, or injection of desired cell populations, for example, stem cells or stem cell-derived cell populations. The ePACs may be used to replace or enhance existing tissues so that the tissue may become a newly altered tissue or may be bound with a biological tissue or structure. Further, in therapeutic protocols of using stem cells derived from tissues other than the umbilical cord, the stem cells may be replace by the ePACs of the present disclosure.

The cell therapeutic agent may be prepared as an injectable formulation. In this case, ingredients commonly known for the formulation may be used, and a common method may be used for the formulation.

### Advantageous Effects of the Invention

Enhanced postnatal adherent cells according to an aspect may be free from ethical issues and easily obtained because a tissue that is discarded from the mother after childbirth is donated, and the enhanced postnatal adherent cells may have characteristics of fetus-derived cells to show excellent proliferation ability and may release a large amount of proteins which are known to be useful for neurological diseases, immune diseases, or vascular diseases. Accordingly, the enhanced postnatal adherent cells may be suitable for compositions of therapeutic agents, and thus utilized in the development of cell therapeutic agents.

### DESCRIPTION OF THE DRAWINGS

FIG. 1 shows results of analyzing Oct4a and Nanog expression patterns of embryonic stem cells (ES) and enhanced postnatal adherent cells (ePACs) by immunocytochemistry;

FIG. 2 shows a morphology of ePACs observed by using an inverted microscope (Eclipse TS100 (Nikon) at 100× magnification;

FIG. 3 shows immune-modulating ability of ePACs, in which the graph represents inhibition rates of T cell proliferation when monocytes isolated from the peripheral blood were directly or indirectly co-cultured with ePACs;

FIG. 4 shows immune-modulating ability of ePACs according to subculturing, in which the graph represents inhibition rates of T cell proliferation when monocytes isolated from the peripheral blood were directly or indirectly co-cultured with ePACs at passage 1 (P1), passage 3 (P3), or passage 6 (P6);

FIG. 5 shows multipotency of ePACs of the present disclosure, in which the ePACs of the present disclosure differentiated to adipocytes, osteocytes, or chondrocytes under both conditions of hypoxia and normoxia;

FIG. 6 shows migration ability of ePACs cultured under hypoxia and normoxia conditions, n=3, * P<0.06;

FIG. 7 shows colony forming ability of ePACs cultured under hypoxia and normoxia conditions, n=3, * P<0.06;

FIG. 8 shows characteristics of ePACs of the present disclosure according to culture passages, in which A represents a cumulative population doubling level (CPDL) according to each cell passage of ePACs, B represents a population doubling level (PDL) according to each cell passage of ePACs, and C represents a cell size of ePACs according to each cell passage;

FIG. 9 shows results of ELISA for analyzing secretion of VEGF, TGF-β1, progranulin, and IL-6 from ePACs at each passage, after examining secretory proteins of ePACs by Ab arrays;

FIG. 10 shows results of ELISA for analyzing secretion of HGF from ePACs at each passage;

FIG. 11 are graphs showing results of flow cytometry for analyzing immunological characteristics of ePACs for HLA-ABC, HLA-DR, and HLA-G and other surface proteins;

FIG. 12 shows results of comparing and analyzing protein expressions of ePACs according to a specific embodiment; and

FIG. 13 shows results of analyzing T cell proliferation-inhibitory effect (A) and neuronal cell-proliferating effect (B) of ePACs according to a specific embodiment.

### MODE OF THE INVENTION

Hereinafter, the present invention will be described in more detail. However, these Examples are for illustrative purposes only, and the scope of the present invention is not intended to be limited by these Examples.

### Example 1. Preparation of Enhanced Postnatal Adherent Cells

1.1. Isolation of Adherent Cells from Amnion Tissue of Placenta

A placenta of a healthy woman who had normally delivered was used, after an informed consent form was signed by the woman who had been given information about the research. A chorionic plate membrane was pulled and peeled off from a placental tissue collected during normal placenta delivery. The removed chorionic plate membrane was washed with Ca/Mg free DPBS containing gentamicin twice or five times to remove blood, and then a chorionic plate was scraped and removed by using a slide glass. The remaining amnion was divided into pieces as small as possible using surgical scissors such that the pieces was in a size of about 1 mm to about 5 mm, and 20 ml of an enzyme reaction solution (enzyme mixture) was added to the small tissue pieces and allowed to react in a shaking incubator at 37° C. and 200 rpm for 15 minutes. Components and concentrations of the used enzyme reaction solution are given in the following Table 1. To inactivate the enzyme reaction solution, 2 ml of FBS was added at a ratio of 1:10, and the reaction solution was centrifuged at 1,500 rpm for 3 minutes, and then a supernatant was transferred to a new tube. This procedure was repeated twice with respect to the remaining tissues. From the dissolved tissue, amniotic cells were isolated using a 100-μm cell strainer.

TABLE 1

| Component | Concentration | | Available source |
|---|---|---|---|
| HBSS (Hank's Balanced | — | | Invitrogen |
| Salt Solution) | | | |
| Trypsin | 1.8 | mg/ml | Sigma |
| Dispase | 1 | U/ml | Invitrogen |
| Collagenase I | 1.2 | mg/ml | Invitrogen |
| DNase I | 25 | μg/ml | Rhoche |

1.2. Culture of Isolated Adherent Cells

(1) Culture Under Hypoxia Condition

The amniotic cells isolated in Example 1.1 were centrifuged and a supernatant was removed therefrom. Cell pellets were suspended in a PS-CM medium, and seeded in a T-flask, and then cultured at 37° C. under a hypoxia condition (CO₂ 5%, O₂ 3%). The cells were cultured until cell colonies were formed to occupy 50%˜80% of the bottom area of T-flask. Every 3 days to 4 days, PS-CM medium was replaced to remove cells which did not adhere to the bottom of flask. Components of the PS-CM medium are given in the following Table 2. Only cells isolated by treatment of TrypLE, which is an animal component-free (ACF) recombinant enzyme (Invitrogen), in a 37° C. incubator for a short time (3 minutes) at a first passage, were used to increase purity of amnion-derived adherent cells.

TABLE 2
Components of PC-CM medium

| Component | Concentration | | Available source |
|---|---|---|---|
| MEM alpha GlutaMAX | | | Invitrogen |
| Fetal Bovine Serum (FBS) | 10% | | Invitrogen |
| Fibroblast growth factor 4 | 25 | ng/ml | Peprotech |
| (FGF4) | | | |
| Heparin | 1 | μg/ml | Sigma |
| Gentamicin | 50 | μg/ml | Invitrogen |

(2) Culture Under Normoxia Condition

The amniotic cells isolated in Example 1.1 were cultured in the same manner as in the culture under hypoxia condition in Example 1.2.(1) except that an oxygen concentration was changed during culture. The oxygen concentration during culture was 21%, and the same PC-CM medium as in Example 1.2.(1) was used.

### Example 2. Characterization of Prepared Enhanced Postnatal Adherent Cells

2.1. Examination of Cell Morphology

Morphology and culture characteristics of the enhanced postnatal adherent cells cultured in Example 1 were observed by using an inverted microscope (Eclipse TS100 (Nikon)) at 100× magnification.

FIG. 2 is a microscopic image of the cells, and the enhanced postnatal adherent cells of the present disclosure had a morphology specific to fibroblasts with irregular protrusions and proliferated while adhering to a plastic ware. The enhanced postnatal adherent cells of the present disclosure were smaller than known placenta-derived mesenchymal stem cells, and had a homogenous cell morphology.

2.2. Analysis of Surface Antigen Expression

(1) Flow Cytometric Analysis

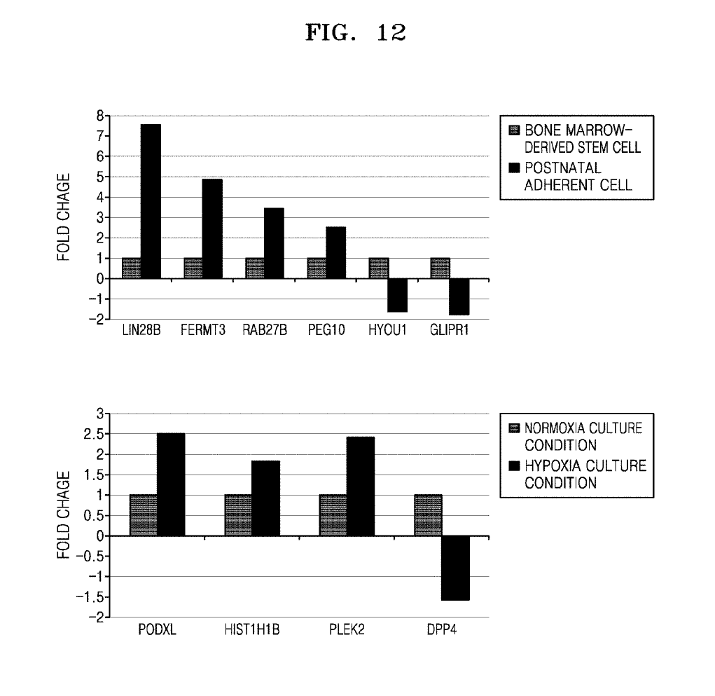

To analyze characteristics of the prepared amnion-derived adherent cells, each 1×10⁶ of the cells cultured under hypoxia and normoxia conditions of Example 1 were collected in a 1.5 ml tube, and reacted with fluorescent-labeled CD1a, CD3, CD8a, CD11c, CD14, CD16, CD19, CD31, CD34, CD40, CD44, CD45, CD56, CD61, CD73, CD80, CD86, CD90, CD105, CD106, CD130, CD166, CD200, CD321, CD338, PDGFRα, PDGFRβ, TRA-1-60, HLA-DR, HLA-G, HLA-ABC, MIC A/B and SSEA4 antibodies, respectively. Cell-specific surface antigen expressions were analyzed by using a flow cytometer (Facs Caliber, BD science), and the results are shown in FIG. 11.

As a result, all of the cells cultured under hypoxia and normoxia conditions showed negative characteristics for CD1a, CD3, CD8a, CD11c, CD16, CD19, CD31, CD34, CD40, CD45, CD56, CD80, CD86, PDGFα, TGA-1-60, HLA-DR, and HLA-G, and positive characteristics for CD44, CD61, CD73, CD90, CD105, CD130, CD166, CD200, CD321, CD338, SSEA4, PDGFRβ, and HLA-ABC. Meanwhile, 10% or more of the cells showed CD106 expression and 70% or more of the cells showed CD200 expression (see FIG. 11).

Oct4 and Nanog expression patterns in the cells cultured in Example 1 were examined by immunocytochemistry (ICC). Embryonic stem cells (ES) were used as a positive control group and Oct4 and Nanog expression patterns at P1 and P6 of the cells were analyzed.

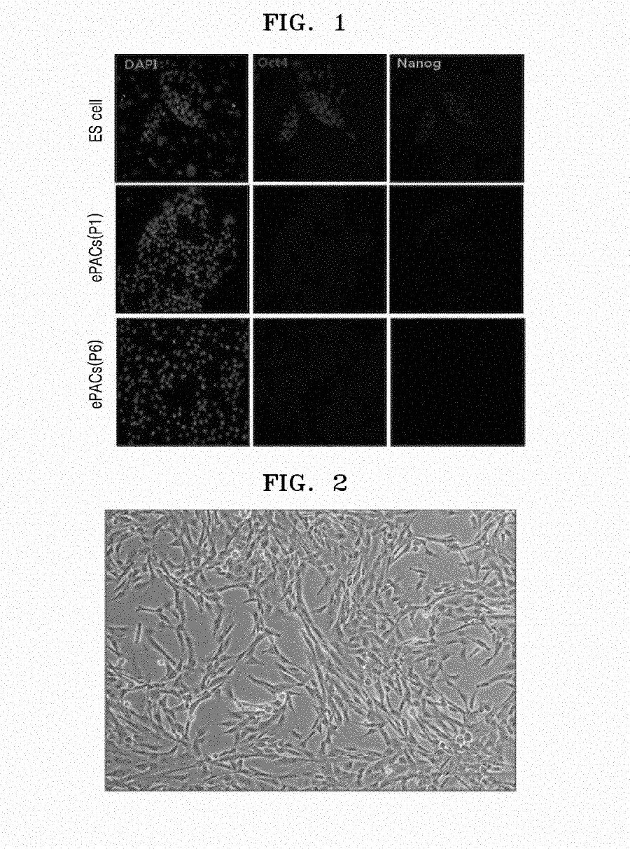

Specifically, the collected cells were washed with DPBS three times, and then fixed in PBS containing 4% paraformaldehyde for 10 minutes. The cells were washed with DPBS three times, and allowed to react with 0.2% Triton X-100 solution at room temperature for 10 minutes, and washed with DPBS three times. Blocking was performed by using 10% normal goat serum at room temperature for 30 minutes. Stem cell markers, Oct4 and Nanog, which are primary antibodies, were added and allowed to react overnight at 4° C. in the dark. Thereafter, the cells were washed with DPBS three times, and secondary goat antibodies were added thereto, and allowed to react in the dark for 1 hour. Lastly, the cells were washed with DPBS three times, and observed under a fluorescence microscope (FIG. 1).

As a result, all the enhanced postnatal adherent cells of the present disclosure cultured under hypoxia and normoxia conditions showed negative characteristics for Oct4 and Nanog which function to maintain progenitor cell characteristics of embryonic stem cells (FIG. 1), indicating loss of possibility of teratoma formation. Accordingly, it is expected that the cells of the present disclosure have high stability when they are applied to cell therapeutic agents.

### Example 3. Profiling and Quantification Analysis of Secretory Proteins of Enhanced Postnatal Adherent Cells

3.1. Profiling of Secretory Proteins

Profiling of secretory proteins was performed in order to analyze secretory proteins of the cells prepared in Example 1. In detail, the adherent cells cultured under normoxia and hypoxia conditions until confluency reached 90% were allowed to secrete secretory proteins in serum-free MEM alpha GlutaMAX (Invitrogen), and secretory proteins were concentrated at a concentration of 1 mg/ml. The concentrate was reacted with a membrane (human antibody array (Raybio)) capable of analyzing 504 kinds of secretory proteins, and fluorescence was developed to examine proteins secreted from the adherent cells. As shown in the following Table 3, a total of 54 kinds of proteins were found to be secreted.

TABLE 3

| | Cytokine |
|---|---|
| 1 | Thrombospondin (TSP) |
| 2 | EDA-A2 |
| 3 | IGFBP-rp1/IGFBP-7 |
| 4 | Thrombospondin-1 |
| 5 | MMP-1 |
| 6 | HGF |
| 7 | IL-8 |
| 8 | sgp130 |
| 9 | WIF-1 |
| 10 | IL-6 |
| 11 | TIMP-2 |
| 12 | GRO |
| 13 | Latent TGF-beta bp1 |
| 14 | GDF-15 |
| 15 | sFRP-4 |
| 16 | IL-19 |
| 17 | Kremen-2 |
| 18 | TGF-beta RIII |
| 19 | M-CSF |
| 20 | MSP alpha Chain |
| 21 | MIP 2 |
| 22 | TNF RI/TNFRSF1A |
| 23 | MCP-3 |
| 24 | Galectin-3 |
| 25 | MCP-1 |
| 26 | sFRP-1 |
| 27 | TGF-beta RI/ALK-5 |
| 28 | IL-15 R alpha |
| 29 | ENA-78 |
| 30 | IL-7 |
| 31 | SPARC |
| 32 | VEGF |
| 33 | Inhibin B |
| 34 | IGFBP-3 |
| 35 | Nidogen-1 |
| 36 | EMAP-II |
| 37 | Progranulin |
| 38 | MIF |
| 39 | IL-3 |
| 40 | IGFBP-6 |
| 41 | TIMP-1 |
| 42 | IGF-II R |
| 43 | Activin C |
| 44 | Smad 4 |
| 45 | Decorin |
| 46 | Dkk-1 |
| 47 | MIP-1a |
| 48 | FGF-7/KGF |
| 49 | Follistatin |
| 50 | Angiostatin |
| 51 | MMP2 |
| 52 | MMP10 |
| 53 | TRAIL R2 |
| 54 | MMP3 |

3.2. Quantification Analysis of Secretory Proteins

Among the secretory proteins, VEGF, TGF-β1, progranulin, HGF, and IL-6 which are known as useful proteins for neurological diseases, immune diseases, or vascular diseases were found to be secreted at a large amount, and quantification analysis thereof was performed according to a donor and a passage.

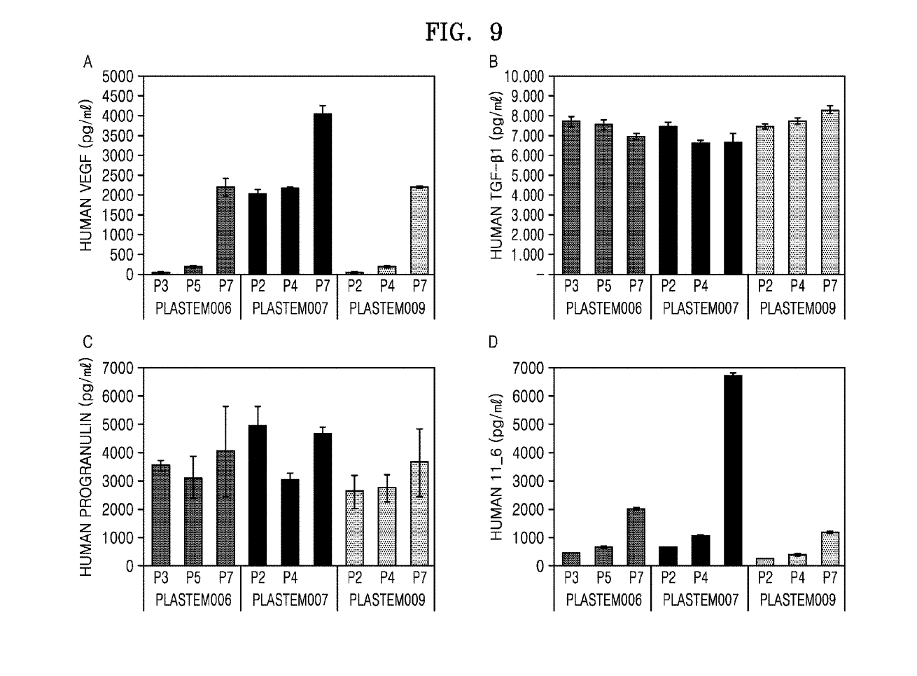

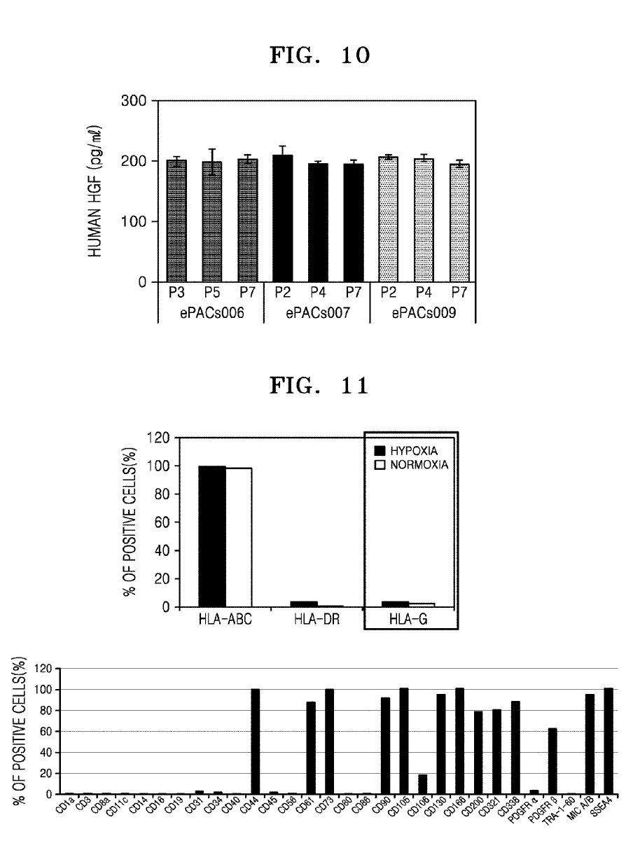

The amounts of the five secretory proteins were analyzed by an enzyme-linked immunosorbent assay (ELISA). An equal number of the cells at each passage (see FIGS. 9 and 10) was seeded in a 6-well plate and cultured for 1 day. Then, the medium was replaced by serum-free MEM alpha GlutaMAX (Invitrogen) and cultured for 1 days. This culture was used as a sample. As in the following Table 4, respective ELISA kits were used, and among them, TGF-β1 includes a pretreatment process of the sample. All were measured at 450 nm by using a microplate reader Epoch (BioTek Inc.), and analyzed by using a Gen5 (2.00) software, and the results are shown in FIGS. 9 and 10.

TABLE 4

| ELISA Kit | Product No. | Available source |
|---|---|---|
| Human VEGF | DVE00 | R&D system |
| Human TGF-β1 | DB100B | R&D system |
| Human progranulin | DPGRN0 | R&D system |
| Human HGF | SEA047Hu | Uscn life Science Inc. |
| Human IL-6 | D6050 | R&D system |

As a result, VEGF secretion was increased with increasing passage, and TGF-B1 was constantly secreted regardless of the donor or passage. Further, progranulin which is a protein useful for improvement of neurological diseases showed no great difference in the secretion according to the donor or passage. IL-6 expression levels were increased with increasing passage, and showed a difference according to the donor. HGF was secreted at a small amount and there was no difference according to the donor or passage.

### Example 4. Profiling and Quantification Analysis of Secretory Proteins of Enhanced Postnatal Adherent Cells

4.1. Analysis of Multipotency

Differentiation ability of the adherent stem cells isolated in Example 1 into adipocytes, osteocytes, or chondrocytes was tested under hypoxia and normoxia conditions.

4.1.1. Analysis of Adipocyte Differentiation Ability

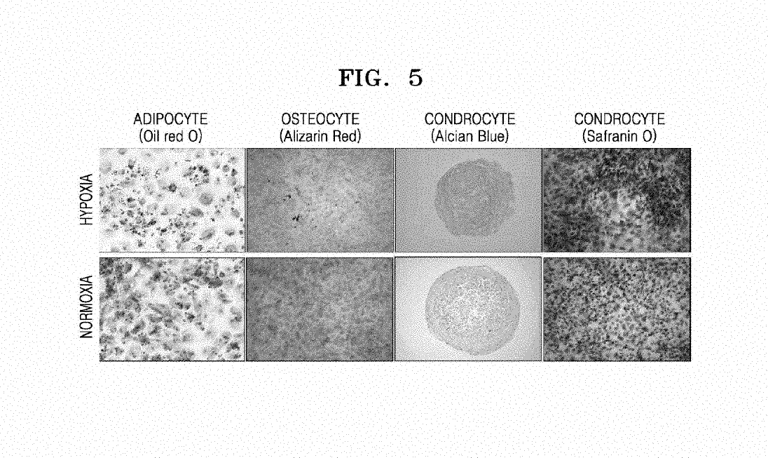

The cells at passage 7 cultured according to 1.2 (1) and 1.2 (2) of Example 1 were cultured in an adipogenesis differentiation medium (StemPro® Adipogenesis Differentiation Kit, Life Technology) for 2 weeks while replacing the medium every three days. Then, the culture medium was removed and cells were washed with Ca/Mg free DPBS, and reacted with 4% paraformaldehyde at room temperature for 15 minutes. The cells were washed with 60% isopropanol and then reacted with Oil Red 0 for 10 minutes. Then, the cells were washed with purified water, and adipocytes were observed under a microscope (FIG. 5).

4.1.2. Analysis of Osteocyte Differentiation Ability

The cells prepared in 1.2 (1) and 1.2 (2) of Example 1 were cultured in an osteogenesis differentiation medium (StemPro® Osteogenesis Differentiation Kit, Life Technology) for 2 weeks while replacing the medium every three days. Then, the culture medium was removed and cells were washed with Ca/Mg free DPBS, and reacted with 4% paraformaldehyde at room temperature for 15 minutes. After reaction, the cells were washed with purified water, and reacted with a 1% silver nitrate solution at room temperature for 5 minutes. The cells were washed with purified water and then reacted with a 5% sodium thiosulfate solution at room temperature for 5 minutes. Then, the cells were washed with purified water, and reacted with a 0.1% nuclear fast red solution at room temperature for 5 minutes. Then, the cells were washed with purified water, and calcium deposition sample was observed under a microscope (FIG. 5).

4.1.3. Analysis of Chondrocyte Differentiation Ability

2×10⁵ of the cells prepared in 1.2 (1) and 1.2 (2) of Example 1 were put in a 15 ml tube, and centrifuged at 1,500 rpm for 5 minutes. The supernatant was discarded, and only cells were cultured in a chondrogenesis differentiation medium (Stem Pro® Chondrogenesis Differentiation Kit, Life Technology) with a lid closed loosely for 3 weeks while replacing the medium every three days. Then, the cell mass was made into a paraffin block and cut in a thickness of 2 mm on a slide glass, followed by Alcian blue staining. Thereafter, chondrocytes stained with blue color were analyzed by optical microscope and the results are shown in FIG. 5.

As shown in FIG. 5, the enhanced postnatal adherent cells of the present disclosure were found to differentiate into adipocytes, osteocytes, or chondrocytes under hypoxia and normoxia conditions.

4.2. Characterization According to Growth

Adherent cells were isolated from the amnion and cultured in the same manner as in the culture method under hypoxia condition of Example 1. To compare doubling times of adherent cells subcultured for 1 passage to 15 passages, each same number of cells was seeded in a 6-well plate, and cells were harvested when they occupied 70%˜80% of the bottom area of the plate, and the number of cells was measured. The measurement was performed in triplicate. 10 μl of a cell suspension was mixed with 10 μl of trypan blue, and 10 μl thereof was counted by using a hemocytometer. The doubling time, which is a time it takes for a cell to double, was calculated using the total number of cells and the time when the number was measured.

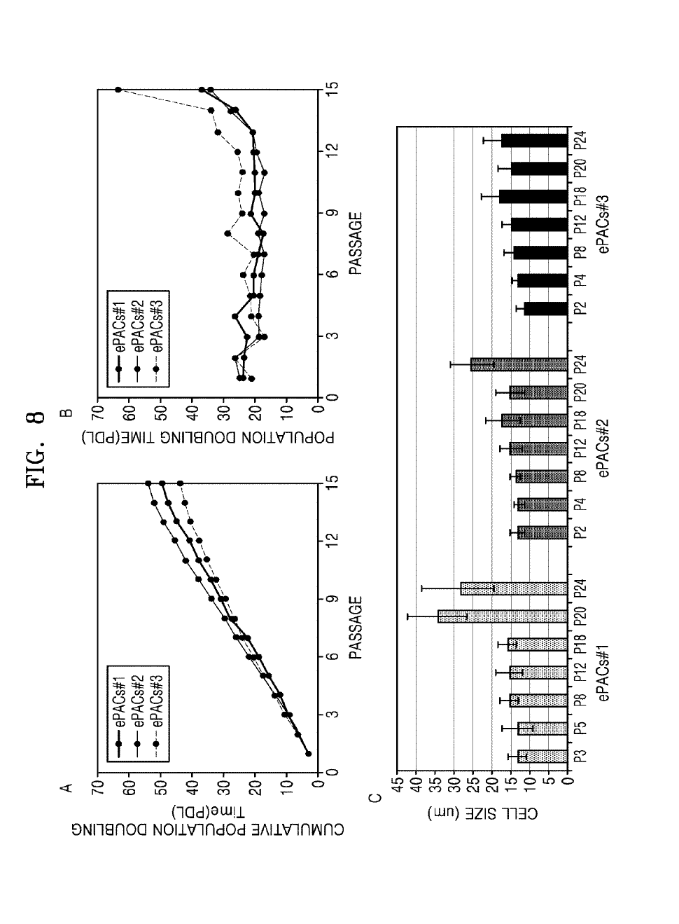

As shown in FIGS. 8A and 8B, the characteristics were maintained up to about passage 12, and the doubling time of about 20 hours was maintained.

Further, the adherent cells were cultured to passages 24, and the sizes of the cells were measured. Three cell samples were used, and the sizes of the cells were measured by using an Autocell counter. All of the three adherent cells showed similar growth patterns. As shown in FIG. 8C, the cell size was about 20 μm or less until p18, and 13 μm˜14 μm at early passages. Meanwhile, as the passage number increased, the cell size increased.

4.3. Analysis of Cell Migration Ability and Colony Forming Ability

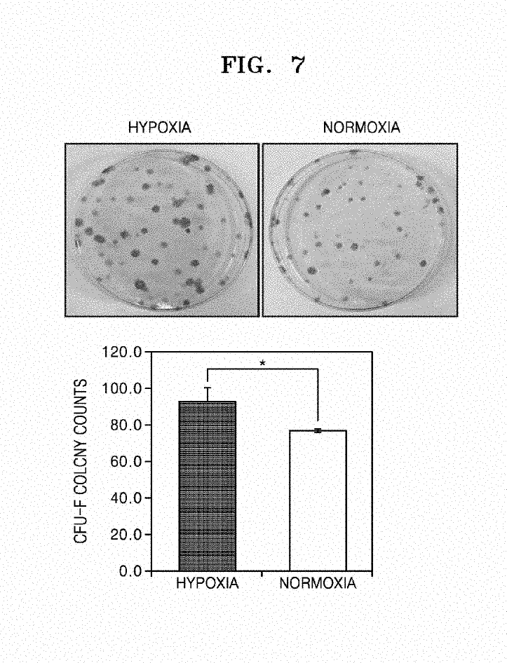

To analyze cell's ability to migrate to damaged tissues, a transwell was used to examine cell's migration ability. The upper surface of the transwell was coated with 0.1% gelatin at 37° C. Each 5×10⁵ of the cells cultured under hypoxia and normoxia conditions in Example 1 were suspended in serum-free medium, and seeded to the upper chamber of the transwell insert. PS-CM containing chemokines was added to a culture medium of the lower chamber. Cells were cultured in an incubator overnight. Cells that migrated through the transwell was subjected to giemsa's staining, and the number of cells was counted under an optical microscope to confirm cell migration, and results are shown in FIG. 7.

It was confirmed that all of the cells cultured under hypoxia and normoxia conditions had migration ability and the migration ability of the adherent cells cultured under the hypoxia condition was improved twice or higher, as compared with that of the cells cultured under the normoxia condition.

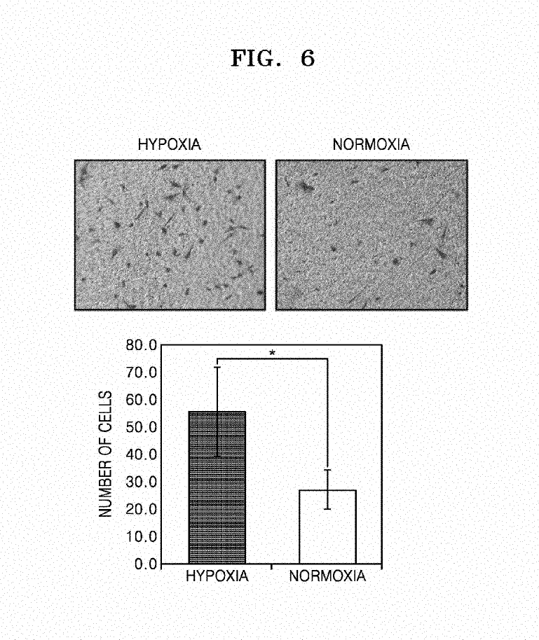

Further, the colony forming ability of the enhanced postnatal adherent cells cultured under normoxia and hypoxia conditions was examined by CFU analysis. To analyze cell's colony forming ability, cells were cultured in a 100 mm culture dish for 10 days to 14 days, and colony formation was examined under a microscope. Next, the cells were washed with DPBS, and then 2 mL to 3 mL of a mixed solution of glutaraldehyde and crystal violet was added to cells in the dish and stained for 30 minutes. The cells were carefully washed with sterile water and the number of colonies was counted under a microscope, and presented as mean values to analyze results. As shown in FIG. 6, colony formation (CFU) of the adherent cells cultured under the hypoxia condition was increased about 1.2 times, as compared with that of the cells cultured under the normoxia condition. In addition, the size of the colony was also increased (FIG. 7B).

### Example 5. Examination of Functions of Enhanced Postnatal Adherent Cells

5.1. Examination of Immune-Modulating Function

When monocytes isolated from the peripheral blood are activated with PHA, T cells proliferate. When T cell proliferation is inhibited by co-culturing of the monocytes and the adherent cells of the present disclosure, it may be assured that the adherent cells of the present disclosure have immune-modulating function.

Monocytes were isolated from the donated peripheral blood by using Ficoll, and used. 50,000 of the cells prepared in Example 1 were seeded in a 24 well plate, and adhered to the plate in an incubator at 37° C. overnight. Thereafter, to activate monocytes stained with CFSE, 10 μg/ml of PHA mixed with a culture medium was added. 4×10⁵ cells were added and co-cultured directly or indirectly for 5 days. At this time, the direct co-culture was performed by co-culturing the monocytes and the enhanced postnatal adherent cells in the 24 well, and the indirect co-culture was performed by adding the monocytes in the upper chamber of the Transwell. Thereafter, the monocytes were recovered, and T cell proliferation rates were analyzed by a flow cytometer (FACS Caliber, BD science).

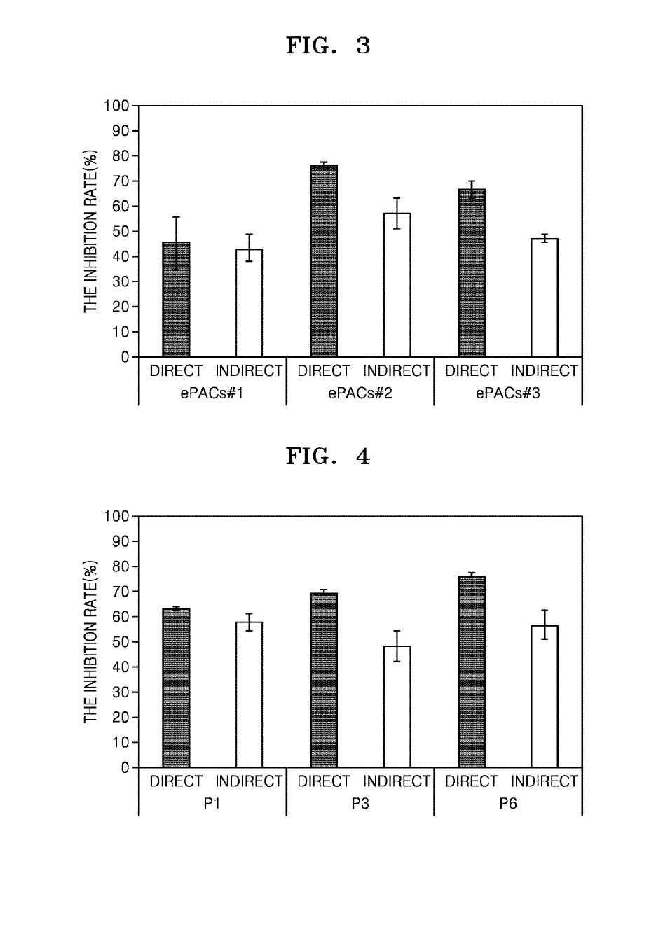

As a result, it was confirmed that the adherent cells of the present disclosure had the immune-modulating function, and when the adherent cells were co-cultured with monocytes directly or indirectly, all the cells showed the immune-modulating function. Specifically, when analysis was performed according to the donor, the adherent cells derived from all donors showed 40% or more of inhibition rate (FIG. 3). Further, when analysis was performed according to the passage, direct co-culture of the peripheral blood monocytes and the adherent cells showed improved inhibition as the passage increased (FIG. 4).

5.2. Analysis of Nerve Regeneration Effect

A nerve regeneration effect of the enhanced postnatal adherent cells isolated and cultured in Example 1 was analyzed.

In detail, MEM and conditioned medium of the enhanced postnatal adherent cells were collected and prepared as samples. Thereafter, nerve cells (SH-SY5Y) were inoculated in a 96-well plate. When the cells were proliferated for about 1 day, MEM and the culture medium of the enhanced postnatal adherent cells were added thereto, respectively and cultured for 4 days. A reagent of Cyto X™ Cell viability assay kit (WST-1) was added to the medium at an amount of 10% thereof, and allowed to react in an incubator for 2 hours to 3 hours. Thereafter, proliferation rates of SH-SY5Y cells were analyzed at 450 nm by using a microreader, and the results are shown in FIG. 13.

FIG. 13 shows results of analyzing the nerve regeneration effect of enhanced postnatal adherent cells according to a specific embodiment. As shown in FIG. 13, when the proliferation rate of the nerve cells cultured in MEM medium was taken as 100%, the proliferation rate of the nerve cells (represented by ‘ePACs culture medium’) cultured in the culture medium of the enhanced postnatal adherent cells (ePACs) was about 280%, which is 2.5 times or higher, as compared with that of the control group.

The result suggests that the enhanced postnatal adherent cells according to a specific embodiment have the immune disease-improving effect and the nerve regeneration effect, and therefore, the postnatal adherent cells of the present disclosure may be applied to the treatment of neurodegenerative diseases.

### Example 6. Comparison of Gene and Protein Expression Characteristics of Enhanced Postnatal Adherent Cells

6.1. Comparison of Gene and Protein Expression Between Enhanced Postnatal Adherent Cells and Mesenchymal Stem Cells (BMMSC) at mRNA and Protein Levels

A difference in gene expression was compared between the placenta-derived adherent cells of the present disclosure and BMMSCs.

RNAs and proteins were extracted from the enhanced postnatal adherent cells and bone marrow-derived mesenchymal stem cells (BMMSCs) (Available source: Cambridge Univ).

The extracted total RNAs were isolated and amplified by using a Target Amp-Nano Labeling Kit for an Illumina Expression BeadChip (EPICENTRE, Madison, USA). 500 ng of total RNA and T7 oligo(dT) primer were used to synthesize cDNA, and in vitro transcription was performed by using biotin-UTP to prepare biotin-labeled cRNA. The prepared cRNA was quantified by using a NanoDrop. The prepared cRNA was hybridized onto a HT-12 v4.0 expression beadchip. After hybridization, to remove non-specific hybridization, the DNA chip was washed with a wash buffer of Illumina Gene Expression System (Illumina), and the washed DNA chip was labeled with a fluorescent streptavidin-Cy3 (Amersham). The fluorescent-labeled DNA chip was scanned by using a confocal laser scanner (Illumina) and fluorescent data present in each spot were saved with TIFF image files. TIFF image files were analyzed by BeadStudio version 3 (Illumina) to quantify spot fluorescent intensities. The quantified results were subjected to Gene-Enrichment and functional analysis by using a DAVID (http://david.abcc.ncifcrf.goc/home.jsp) program.

As a result, when genes showing variances in expression level of 2-fold or more were selected, 2635 genes were observed. Among them, expression levels of 1305 genes were increased and expression levels of 1330 genes were decreased in the postnatal adherent cells of the present disclosure, as compared with BMMSCs. Among them, 23 genes showing the greatest difference in the expression level was selected, and shown in the following Table 5. Among them, COL3A1, IGFBP5, PRNP, MT1A, and CCND1 are genes that showed increased expression levels in the postnatal adherent cells, as compared with BMMSCs. COL1A2, COL1A1, TPM2, TAGLN, CALD1, COL6A3, IGFBP7, SPARC, EFEMP1, CYP1B1, CXCL12 and PENK are genes that showed decreased expression levels in the postnatal adherent cells, as compared with BMMSCs.

TABLE 5

| | ProbeID | ACCESSION | SYMBOL | fold change |
|---|---|---|---|---|
| 1 | ILMN_1785272 | NM_000089.3 | COL1A2 | −2.152358 |
| 2 | ILMN_1701308 | NM_000088.3 | COL1A1 | −2.238551 |
| 3 | ILMN_1757604 | NM_213674.1 | TPM2 | −2.183863 |
| 4 | ILMN_1773079 | NM_000090.3 | COL3A1 | 2.13323 |
| 5 | ILMN_1778668 | NM_003186.3 | TAGLN | −3.064249 |
| 6 | ILMN_1803429 | NM_001001391.1 | CD44 | 2.548315 |
| 7 | ILMN_1671703 | NM_001613.1 | ACTA2 | −3.620056 |
| 8 | ILMN_1730487 | NM_033140.2 | CALD1 | −2.928108 |
| 9 | ILMN_1750324 | NM_000599.2 | IGFBP5 | 2.981225 |
| 10 | ILMN_1699829 | NM_001901.1 | CTGF | −3.536551 |
| 11 | ILMN_1737988 | NM_001080121.1 | PRNP | 2.368955 |
| 12 | ILMN_1706643 | NM_057165.2 | COL6A3 | −2.367989 |
| 13 | ILMN_1665865 | NM_001552.2 | IGFBP4 | −3.947149 |
| 14 | ILMN_1691156 | NM_005946.2 | MT1A | 2.091371 |
| 15 | ILMN_2360710 | NM_001018004.1 | TPM1 | −4.322069 |
| 16 | ILMN_2062468 | NM_001553.1 | IGFBP7 | −2.435926 |
| 17 | ILMN_1796734 | NM_003118.2 | SPARC | −2.117699 |
| 18 | ILMN_1686116 | NM_003246.2 | THBS1 | −5.42393 |
| 19 | ILMN_1688480 | NM_053056.2 | CCND1 | 2.499045 |
| 20 | ILMN_2350634 | NM_001039348.1 | EFEMP1 | −2.639375 |
| 21 | ILMN_1693338 | NM_000104.2 | CYP1B1 | −21.273743 |
| 22 | ILMN_1791447 | NM_199168.2 | CXCL12 | −109.091657 |
| 23 | ILMN_1726711 | NM_006211.2 | PENK | −167.876141 |

The extracted total proteins were digested into peptides by FASP which is a tryptic digestion method of using a filter, and for relative quantification, the peptides were subjected to isotope labeling, and mass spectrometry was performed by using an Easy nLC-1000/Q-Exactive system for LC-MS spectrometry.

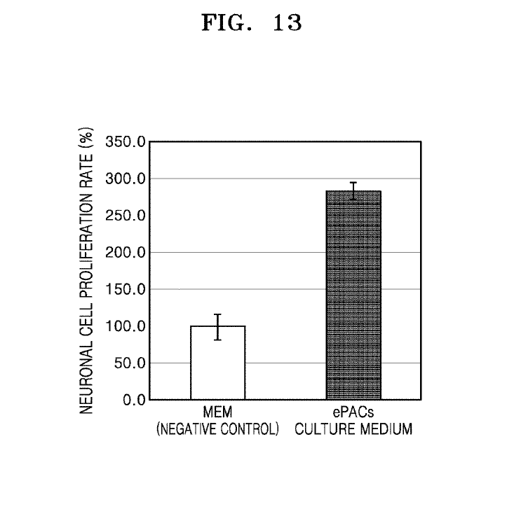

As shown in FIG. 12A, when protein expression was compared between the enhanced postnatal adherent cells and BMMSCs, the postnatal adherent cells showed high expression of LIN28B, FERMT3, RAB27B, and PEG10 and low expression of HYOU1 and GLIPR1, as compared with BMMSCs.

6.2. Comparison of Gene and Protein Expression Between Enhanced Postnatal Adherent Cells Cultured Under Hypoxia Condition and Under Normoxia Condition at mRNA and Protein Levels

A difference in gene expression was compared between the adherent cells cultured under a hypoxia condition and the adherent cells cultured under a normoxia condition.

For comparison, a microarray and a proteomics array were used and a detailed method is the same as in Example 6.1. Further, the enhanced postnatal adherent cells were isolated and cultured in the same manner as in Example 1.

As a result, when genes showing variances in expression level of 2-fold or more were selected, 514 genes were observed. Among them, expression levels of 229 genes were increased and expression levels of 285 genes were decreased in the postnatal adherent cells under the hypoxia condition. Among them, 21 genes showing the greatest difference in the expression level was selected, and shown in the following Table 6. Among them, PGK1, BNIP3, TPI1, ERRFI1, LOC644774, SLC2A3, PLIN2, and TUBB2B were increased and ALDH1A3, SERPINE1, IL6, AKR1B1, NQO1, PAPPA, TAGLN, TGM2, IL1B, IL8, and EFEMP1 were decreased under the hypoxia condition.

TABLE 6

| | Probe ID | ACCESSION | SYMBOL | fold change |
|---|---|---|---|---|
| 1 | ILMN_1755749 | NM_000291.2 | PGK1 | 2.191413 |
| 2 | ILMN_1724658 | NM_004052.2 | BNIP3 | 2.799374 |
| 3 | ILMN_1707627 | NM_000365.4 | TPI1 | 2.075759 |
| 4 | ILMN_1665510 | NM_018948.2 | ERRFI1 | 3.446231 |
| 5 | ILMN_2139970 | NM_000693.1 | ALDH1A3 | −2.278008 |
| 6 | ILMN_1744381 | NM_000602.1 | SERPINE1 | −2.722234 |
| 7 | ILMN_1699651 | NM_000600.1 | IL6 | −2.278956 |
| 8 | ILMN_1701731 | NM_001628.2 | AKR1B1 | −2.365849 |
| 9 | ILMN_1779258 | XM_927868.1 | LOC644774 | 3.295901 |
| 10 | ILMN_1720282 | NM_000903.2 | NQO1 | −2.35445 |
| 11 | ILMN_1721770 | NM_002581.3 | PAPPA | −2.210303 |
| 12 | ILMN_1778668 | NM_003186.3 | TAGLN | −2.595073 |
| 13 | ILMN_1705750 | NM_004613.2 | TGM2 | −2.291753 |
| 14 | ILMN_1775708 | NM_006931.1 | SLC2A3 | 3.072693 |
| 15 | ILMN_1775501 | NM_000576.2 | IL1B | −2.134024 |
| 16 | ILMN_2138765 | NM_001122.2 | PLIN2 | 2.763918 |
| 17 | ILMN_2184373 | NM_000584.2 | IL8 | −4.543316 |
| 18 | ILMN_1881909 | BU536065 | | −2.996621 |
| 19 | ILMN_2350634 | NM_001039348.1 | EFEMP1 | −2.33499 |
| 20 | ILMN_1807439 | NM_000693.2 | ALDH1A3 | −2.783309 |
| 21 | ILMN_1680874 | NM_178012.3 | TUBB2B | −4.767344 |

As shown in FIG. 12, when protein expression was compared between the postnatal adherent cells cultured under the hypoxia condition and the postnatal adherent cells cultured under the normoxia condition, the postnatal adherent cells cultured under the hypoxia condition showed high expression of PODXL, HIST1H1B, and PLEK2 and low expression of DPP4, as compared with the postnatal adherent cells cultured under the normoxia condition.

1. An enhanced postnatal adherent cell having one or more characteristics selected from the group consisting of (a) to (h):
(a) maintaining a morphology of adherent fibroblasts during subculture;
(b) having ability to differentiate into adipocytes, osteocytes, or chondrocytes;
(c) secreting one or more proteins selected from the group consisting of VEGF, TGF-β1, IL-6, progranulin, follistatin, angiostatin, MMP2, MMP10, TRAIL R2, and MMP3;
(d) having surface antigen characteristics of HLA-G−, CD34−, MIC A/B+, CD200+, CD61+, CD130+, CD321+, SSEA4+, and CD338+;
(e) having high expression levels of one or more genes selected from the group consisting of COL3A1, IGFBP5, PRNP and MT1A, as compared with bone marrow-derived stem cells;
(f) having low expression levels of one or more genes selected from the group consisting of COL1A1, COL1A2, TPM2, TAGLN, CYP1B1, CXCL12, and PENK, as compared with bone marrow-derived stem cells;
(g) having high expression levels of one or more proteins selected from the group consisting of LIN28B, FERMT3, RAB27B and PEG10, as compared with bone marrow-derived stem cells; and
(h) having low expression levels of HYOU1 or GLIPR1 protein, as compared with bone marrow-derived stem cells.

2. The enhanced postnatal adherent cell of claim 1, having the characteristics of (e), (f), or both, wherein the characteristics of (e) and (f) show that the expression level difference from those of bone marrow-derived stem cells is twice or more, as measured by microarray analysis.

3. The enhanced postnatal adherent cell of claim 1, wherein the cell is derived from amnion.

4. The enhanced postnatal adherent cell of claim 1, wherein when the cell is cultured under a hypoxia condition or under a normoxia condition it has the following characteristics:
the cell cultured under the hypoxia condition has increased expression levels of one or more genes selected from the group consisting of PGK1, BNIP3, TPI1, ERRFI1, LOC644774, SLC2A3, PLIN2, and TUBB2B, as compared with the cell cultured under the normoxia condition;
the cell cultured under the hypoxia condition has decreased expression levels of one or more genes selected from the group consisting of SERPINE1, TAGLN, TGM2, IL-8, and ALDH1A3, as compared with the cell cultured under the normoxia condition;
the cell cultured under the hypoxia condition has increased expression levels of one or more proteins selected from the group consisting of PODXL, HIST1H1B, and PLEK2, as compared with the cell cultured under the normoxia condition; or
the cell cultured under the hypoxia condition has decreased expression level of DPP4 protein, as compared with the cell cultured under the normoxia condition.

5. A population of enhanced postnatal adherent cells, wherein the postnatal adherent cells have the following characteristics:
(a) maintaining a morphology of adherent fibroblasts during subculture;
(b) having ability to differentiate into adipocytes, osteocytes, or chondrocytes;
(c) secreting one or more proteins selected from the group consisting of VEGF, TGF-β1, IL-6, progranulin, follistatin, angiostatin, MMP2, MMP10, TRAIL R2, and MMP3;
(d) having surface antigen characteristics of HLA-G−, CD34−, MIC A/B+, CD200+, CD61+, CD130+, CD321+, SSEA4+, and CD338+;
(e) having a high expression level of one or more genes selected from the group consisting of COL3A1, IGFBP5, PRNP and MT1A, as compared with bone marrow-derived stem cells;
(f) having a low expression level of one or more genes selected from the group consisting of COL1A1, COL1A2, TPM2, TAGLN, CYP1B1, CXCL12, and PENK, as compared with bone marrow-derived stem cells;
(g) having a high expression level of one or more proteins selected from the group consisting of LIN28B, FERMT3, RAB27B and PEG10, as compared with bone marrow-derived stem cells; and
(h) having a low expression level of HYOU1 or GLIPR1 protein, as compared with bone marrow-derived stem cells.

6. The population of enhanced postnatal adherent cells of claim 5, wherein the cells in the population are derived from amnion.

7. The population of enhanced postnatal adherent cells of claim 5, wherein the cells in the population secrete progranulin.

8. The population of enhanced postnatal adherent cells of claim 5, wherein 70% or more of the cells in the population express CD200.

9. A method of preparing an enhanced postnatal adherent cell, comprising reacting an amnion tissue separated from a chorionic plate membrane of placenta with an enzyme mixture, wherein the cell has the following characteristics:
(a) maintaining a morphology of adherent fibroblasts during subculture;
(b) having ability to differentiate into adipocytes, osteocytes, or chondrocytes;
(c) secreting one or more proteins selected from the group consisting of VEGF, TGF-β1, IL-6, progranulin, follistatin, angiostatin, MMP2, MMP10, TRAIL R2, and MMP3;
(d) having surface antigen characteristics of HLA-G−, CD34−, MIC A/B+, CD200+, CD61+, CD130+, CD321+, SSEA4+, and CD338+;
(e) having high expression levels of one or more genes selected from the group consisting of COL3A1, IGFBP5, PRNP and MT1A, as compared with bone marrow-derived stem cells;
(f) having low expression levels of one or more genes selected from the group consisting of COL1A1, COL1A2, TPM2, TAGLN, CYP1B1, CXCL12, and PENK, as compared with bone marrow-derived stem cells;
(g) having high expression levels of one or more proteins selected from the group consisting of LIN28B, FERMT3, RAB27B and PEG10, as compared with bone marrow-derived stem cells; and
(h) having low expression levels of HYOU1 or GLIPR1 protein, as compared with bone marrow-derived stem cells.

10. The method of claim 9, wherein the enzyme mixture comprises one or more selected from the group consisting of trypsin, dispase, collagenase, and DNAase.

11. The method of claim 9, further comprising isolating the enhanced postnatal adherent cell by reacting an animal component-free recombinant enzyme with the cell recovered after reaction of the amnion tissue and the enzyme mixture.

12. An enhanced postnatal adherent cell prepared by the method of claim 9.

13. A composition comprising the enhanced postnatal adherent cell of claim 1.

14. A pharmaceutical composition comprising the enhanced postnatal adherent cell of claim 1 as an active ingredient suitable for treating or preventing a neurodegenerative disease.

15. The pharmaceutical composition of claim 14, wherein the neurodegenerative disease is one or more selected from the group consisting of Alzheimer's disease, concussion, stroke, Parkinson's disease, Pick's disease, Huntington's disease, progressive supranuclear palsy, multiple sclerosis, amyotrophic lateral sclerosis (ALS), dementia, and traumatic brain injury.

16. An agent comprising the enhanced postnatal adherent cell of claim 1.

17. A cell therapeutic agent comprising the enhanced postnatal adherent cell of claim 1 as an active ingredient.

18. (canceled)

19. A method of treating or preventing a neurodegenerative disease, the method comprising administering the enhanced postnatal adherent cell of claim 1, cell populations thereof, or a culture thereof as an active ingredient to a subject in need thereof.
